(19) United States
(12) Patent Application Publication
(10) Pub. No.: US 2007/0049961 A1
(43) Pub. Date: Mar. 1, 2007

(54) Inflation device for use in orthopedic operations

(76) Inventors: Shih-Yu Tsou, Taipei (TW); Shih-Chun Lu, Taipei (TW)

Correspondence Address: BACON & THOMAS, PLLC, 625 SLATERS LANE, FOURTH FLOOR, ALEXANDRIA, VA, 22314, US

(21) Appl. No.: 11/511,284

(22) Filed: Aug. 29, 2006

(30) Foreign Application Priority Data
Aug. 29, 2005 (TW) 94129547

(51) Int. Cl. A61M 29/00

(52) U.S. Cl. 606/192

(57) ABSTRACT

An inflation device suitable for use in the orthopedic operations includes an inflation body, which is a tubular body with an open end and a closed end; and a connector connected to the open end of the inflation body, wherein a pre-formed inflatable chamber is provided at or close to the closed end of the inflation body. Preferably, the tubular body and the pre-formed inflatable chamber are integrally formed.

### FIELD OF THE INVENTION

The present invention provides an inflation device for use in the orthopedic operations, particularly an inflation device with an inflatable chamber.

### BACKGROUND OF THE INVENTION

An inflation operation in a medullary cavity or vertebral body is often performed during orthopedic operations. At present, there are two common inflation techniques used in orthopedic operations: One is a mechanical inflation device (e.g. U.S. Pat. No. 6,676,665). However, the biggest problem associated with said technique is that the cancellous bone fragments produced during the inflation process often dropped into the inflation device. These fragments stuck in the inflation device and prevent the inflation device from recovery into its original shape. In some occasions, the whole inflation device is stuck at the inflation location and unable to be removed. The other type of inflation device currently available is a loading-type inflation device, e.g. products from the Kyphon Company in the U.S.A. The present invention also relates to a loading-type inflation device. The Kyphon Company's products use various types of balloons to meet different needs. The Kyphon company's patents related to the device per se include, e.g., U.S. Pat. Nos. 5,972,015, 6,066,154, 6,235,043, 6,423,083, 6,607,544, 6,623,505, 6,663,647, and 6,716,216, etc.; and related to the method of use (inflation method) include, e.g., U.S. Pat. Nos. 5,972,015, 6,048,346, 6,235,043, 6,248,110, 6,280,456, 6,440,138, 6,607,544, 6,716,216, 6,719,761, and 6726691, etc. In summary, the inflation devices and methods used by the Kyphon Company involve using a high pressure to inject liquid (e.g. water) into a balloon (various types according to different needs) to achieve the objective of pressing the cancellous bone inside a medullary cavity or vertebral body by inflating the balloon. However, these devices or methods have a lot of disadvantages. For example, a balloon needs to be connected to a nozzle. Thus, the balloon might fall off from the nozzle when receiving a high pressure liquid. Furthermore, in order to meet different demands, the balloon needs to be designed with an internal restraint or an external restraint, as shown in the above-mentioned U.S. Pat. No. 6,066,154, or a special inflation device, as shown in the above-mentioned U.S. Pat. No. 6,235,043. These make the production and use of the balloons becoming relatively difficult. The present invention uses an integrally formed inflation body as the main body of an inflation device for use in orthopedic operations. This resolves various disadvantages associated with the drawbacks in the conventional inflation devices mentioned above.

### SUMMARY OF THE INVENTION

A primary objective of the present invention is to provide an inflation device for use in orthopedic operations.

Another objective of the present invention is to provide an inflation device, which includes a tubular body with a closed end and with a pre-formed inflatable chamber.

Still another objective of the present invention is to provide an inflation device, which includes a tubular body with a closed end and with a pre-expanded inflatable chamber.

### BRIEF DESCRIPTION OF THE DRAWINGS

FIGS. 1 to 5 are schematic views showing the inflation devices according to the first to the fifth preferred embodiments of the present invention.

FIGS. 6A and 6B are schematic views showing the inflation device according to the sixth preferred embodiment of the present invention, wherein the connector of the inflation device in FIG. 6A is not assembled and it is assembled in FIG. 6B.

FIGS. 7A and 7B are schematic views showing the inflation device according to the seventh preferred embodiment of the present invention, wherein the connector of the inflation device in FIG. 7A is not assembled and it is assembled in FIG. 7B.

FIGS. 8A and 8B are schematic views showing the inflation device according to the eighth preferred embodiment of the present invention, wherein the connector and the protective tube of the inflation device in FIG. 8A are not assembled and they are assembled in FIG. 8B.

FIGS. 9A to 9C are schematic views showing a production flowchart of the inflation device according to the second preferred embodiment of the present invention.

FIGS. 10A to 10C are schematic views showing a production flowchart of an inflation device according to the ninth preferred embodiment of the present invention.

FIG. 11A to FIG. 11I are schematic views showing the inflation operation flowchart in a vertebral body by using the inflation device of the present invention.

### DETAILED DESCRIPTION OF THE INVENTION

According to the present invention, an inflation device for use in orthopedic operations includes:

an inflation body, which is a tubular body with an open end and a closed end; and

a connector connected to the open end of the inflation body;

wherein a pre-formed inflatable chamber is provided at or close to the closed end of the inflation body, and preferably the inflation body and the pre-formed inflatable chamber are formed integrally.

Said inflation device is a tool used during a surgery operation. Therefore, said device is preferably made of an elastic and harmless material, and more preferably a polymer with bio-compatibility, e.g. polyurethane (PU), silicone rubber, etc.

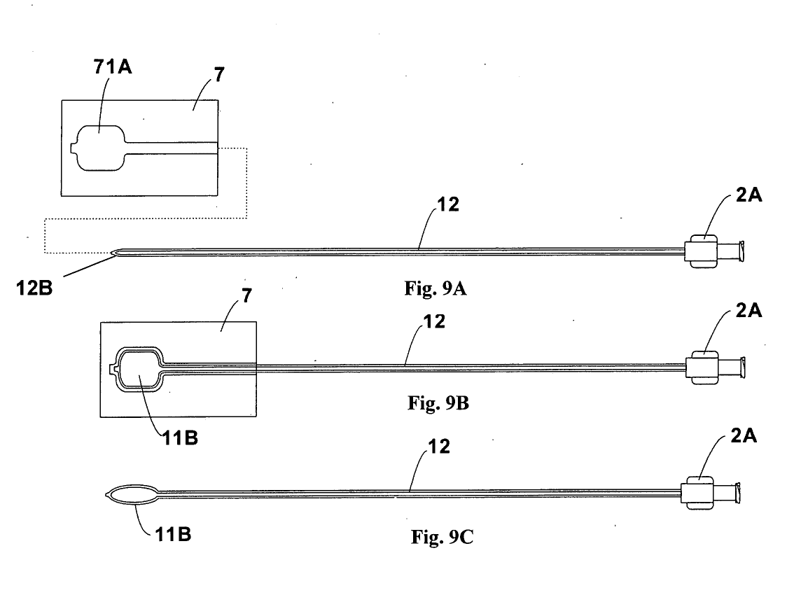

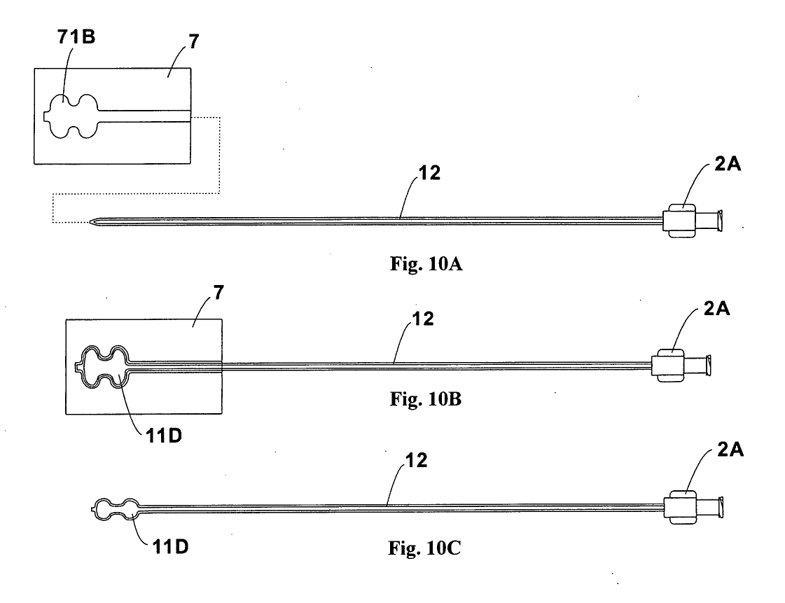

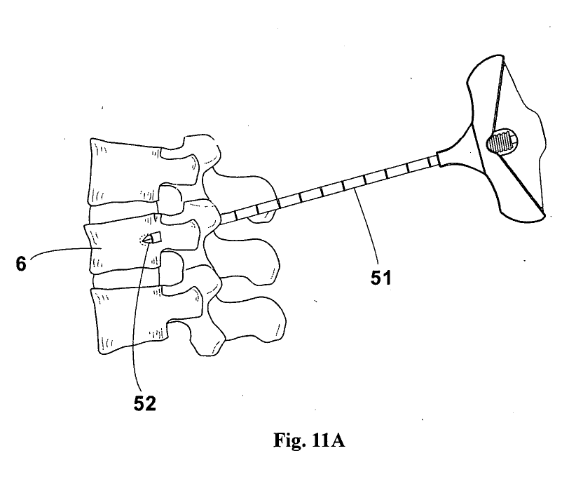

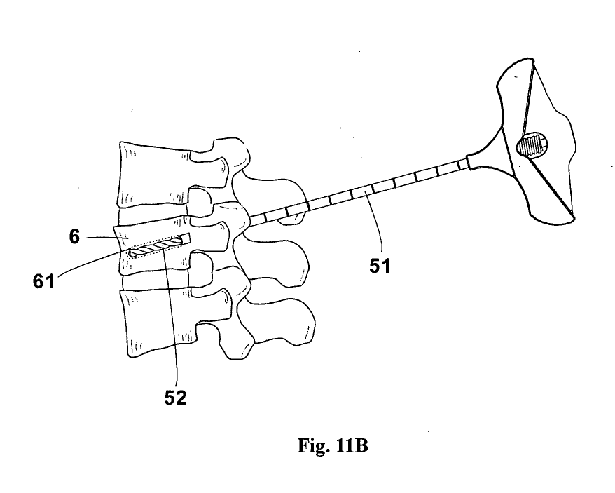

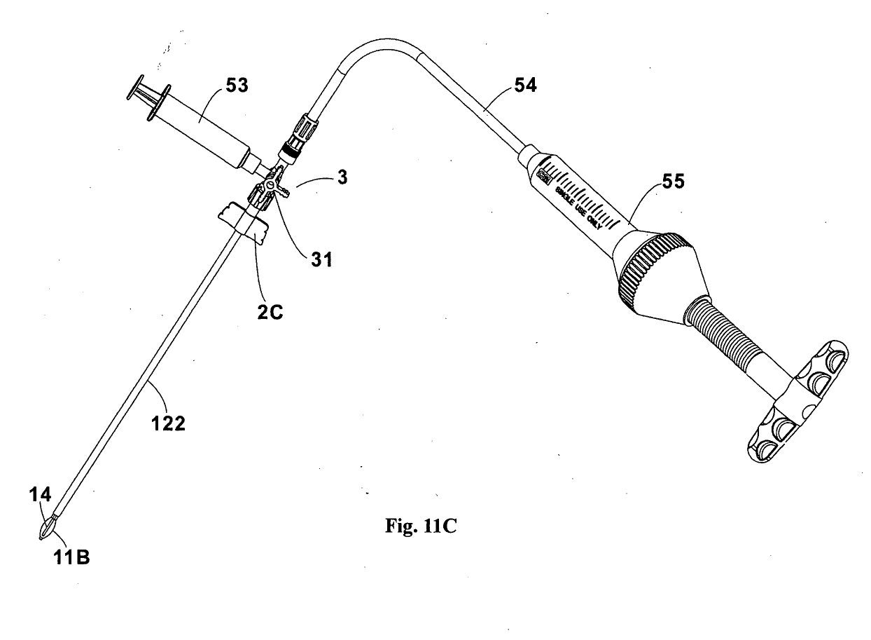

Said pre-formed inflatable chamber is provided at or close to the closed end of the inflation body, and preferably close to the closed end of the inflation body. Said pre-formed inflatable chamber is formed by, for example, casting, molding, pre-expansion casting, and pre-expansion molding, etc., preferably by pre-expansion molding. The pre-expansion molding mentioned herein will be described in the following by referring to FIG. 9A to 9C, and FIG. 10A to 10C.

Optionally, a bent portion is provided on the tubular body near the inflatable chamber in order to be applicable in an operation where a to-be-inflated part is not located in the guided direction of a guide needle.

Optionally, said tubular body is surrounded by a protective tube in order to avoid the occurrence of undesirable inflation during the inflation process. The protective tube and the tubular body can be combined by any conventional combination method, e.g. by inserting the tubular body into the protective tube. The protective tube is detained on the tubular tube by friction.

Optionally, an infusion needle for the convenience of guiding the inflation body is received in said tubular body for guiding the inflation body together with the inflatable chamber into a to-be-inflated portion, and for injecting a liquid (usually water) into the inflatable chamber. The infusion needle and the tubular body can be combined by any conventional method, including simply inserting the infusion needle into the tubular body. The infusion needle is detained in the tubular tube by friction, wherein, the outside diameter of the infusion needle is roughly equal to the inside diameter of the tubular body.

The inflation body and the connector can be combined by any conventional method. Since the joint of the inflation body and the connector is outside a human body, the combination may be accomplished by adhesive.

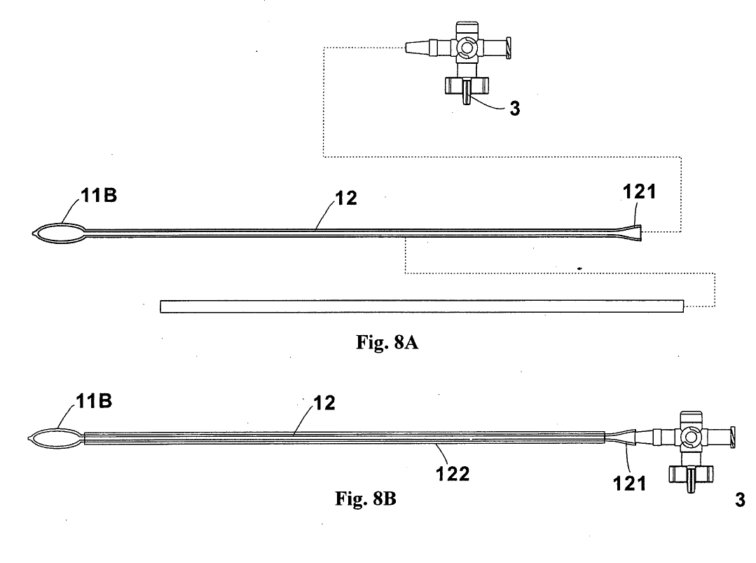

The configuration of said connector is not particularly limited. However, since the connector needs to be externally connected to a three-way switch connector, an infusion gun or a flexible tube, the connector is provided with an external connection port, when necessary, e.g. a threaded portion. Furthermore, the connector can be any conventional connector, e.g. a three-way switch connector shown in FIGS. 8A and 8B.

The following preferred embodiments, together with the related drawings, are disclosed in order to further elaborate the present invention:

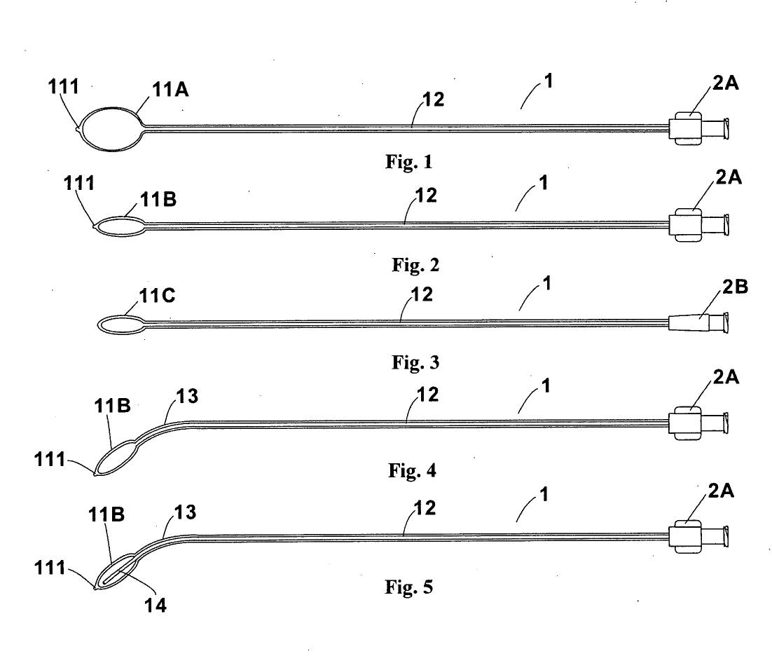

An inflation device constructed according to a first preferred embodiment of the present invention is shown in FIG. 1, which has an inflation body 1, a connector 2A, an inflatable chamber 11A and a tubular body 12 of the inflation body 1. A protective part 111 is provided at the tail end of the inflatable chamber 11A for preventing the guide needle from piercing through the inflatable chamber 11A. The inflation body 1 is integrally formed with a thermal elastic polyurethane tube. The inflatable chamber 11A is for injection inflation. The inflation body 1 and the connector 2A are combined by an instant glue.

FIG. 2 shows an inflation device constructed according to a second preferred embodiment of the present invention, wherein like elements or parts are represented by like numerals. The inflation device shown in FIG. 2 is similar to that shown in FIG. 1, with the proviso that the inflatable chamber 11B is formed by pre-expanded and partially restored to its original shape, which is different from the inflatable chamber 11A in FIG. 1 which is formed by casting.

FIG. 3 shows an inflation device constructed according to a third preferred embodiment of the present invention, wherein like elements or parts are represented by like numerals. The inflation device shown in FIG. 3 is similar to that shown in FIG. 1, with the proviso that the inflation device does not contain the protective part 111 shown in FIG. 2, and the connector 2B has a different shape.

FIG. 4 shows an inflation device constructed according to a fourth preferred embodiment of the present invention, wherein like elements or parts are represented by like numerals. The inflation device shown in FIG. 4 is similar to that shown in FIG. 1, with the proviso that a bent portion 13 is provided between the inflatable chamber 11B and the tube body 12.

FIG. 5 shows an inflation device constructed according to a fifth preferred embodiment of the present invention, wherein like elements or parts are represented by like numerals. The inflation device shown in FIG. 5 is similar to that shown in FIG. 4, with the proviso that an infusion guide needle 14 is inserted in the tubular body 12 and gripped by the tubular body 12. The infusion guide needle 14 may be glued to the open end of the tubular body 12, or the infusion guide needle 14 is connected to the connector 2A by abutting against each other.

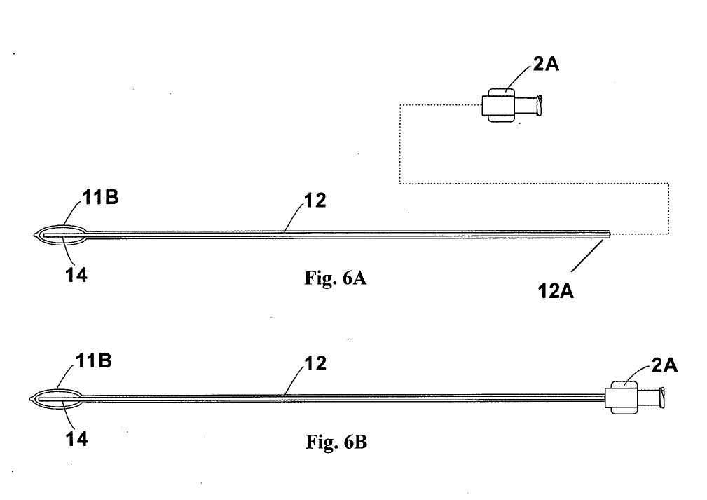

FIG. 6B shows an inflation device constructed according to a sixth preferred embodiment of the present invention, wherein like elements or parts are represented by like numerals. The inflation device shown in FIG. 6B is similar to that shown in FIG. 5, with the proviso that there is no bent portion. FIG. 6A shows the inflation device shown in FIG. 6B prior to the connector 2A being connected to the open end 12A of the tubular body 12.

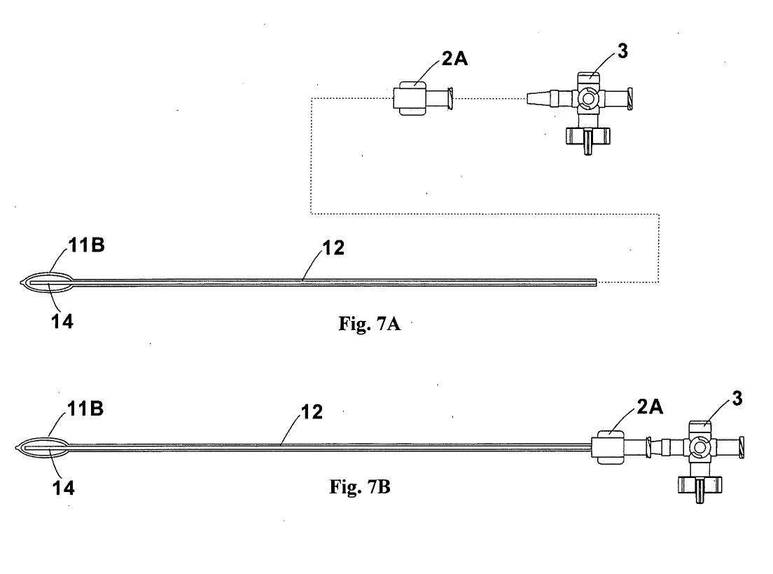

FIG. 7B shows an inflation device constructed according to a seventh preferred embodiment of the present invention, wherein like elements or parts are represented by like numerals. The inflation device shown in FIG. 7B is similar to that shown in FIG. 6B, with the proviso that a three-way switch connector 3 is connected to the connector 2A by glue. FIG. 7A shows the inflation device shown in FIG. 7B prior to the connector 2A and the three-way switch connector 3 being assembled to the tubular body 12.

FIG. 8B shows an inflation device constructed according to an eighth preferred embodiment of the present invention, wherein like elements or parts are represented by like numerals. The inflation device shown in FIG. 8B is similar to that shown in FIG. 2, with the proviso that a three-way switch connector 3 is connected to an expansion open end 121 of the tubular body 12 by glue, and a protective tube 122 is provided to surround the tubular body 12. FIG. 8A shows the inflation device shown in FIG. 8B prior to the protective tube 122 and the three-way switch connector 3 being assembled to the tubular body 12.

A process suitable for preparing the inflation device shown in FIG. 2 are shown in FIGS. 9A to 9C. In FIG. 9A, the closed end 12B of the tubular body 12 is to be introduced into a mold cavity 71A of a mold 7. A high pressure device (not shown) is used to inject water through the connector 2A into the tubular body 12 to inflate a portion of the tubular body near its closed end to conform with the geometry of the mold cavity 71A, as shown in FIG. 9-B, to form a pre-expansion portion (11B), which is partially restored to its original shape to form an inflation chamber 11B as shown in FIG. 9C, after water is withdrawn from the tubular body 12, and the tubular body 12 is removed from the mold.

A process suitable for preparing an inflation device constructed according to a ninth embodiment according to the present invention are shown in FIGS. 10A to 10C, which is similar to the process shown in FIGS. 9A to 9B, wherein like elements or parts are represented by like numerals. In this process the shape of the mold cavity 71B shown in FIG. 10A is different from that of the cavity 71A shown in FIG. 9A. Accordingly, the pre-expansion portion (11D) and an inflation chamber 11D that partially shrinks from the pre-expansion portion also have a different shape from that shown in FIGS. 9B and 9C.

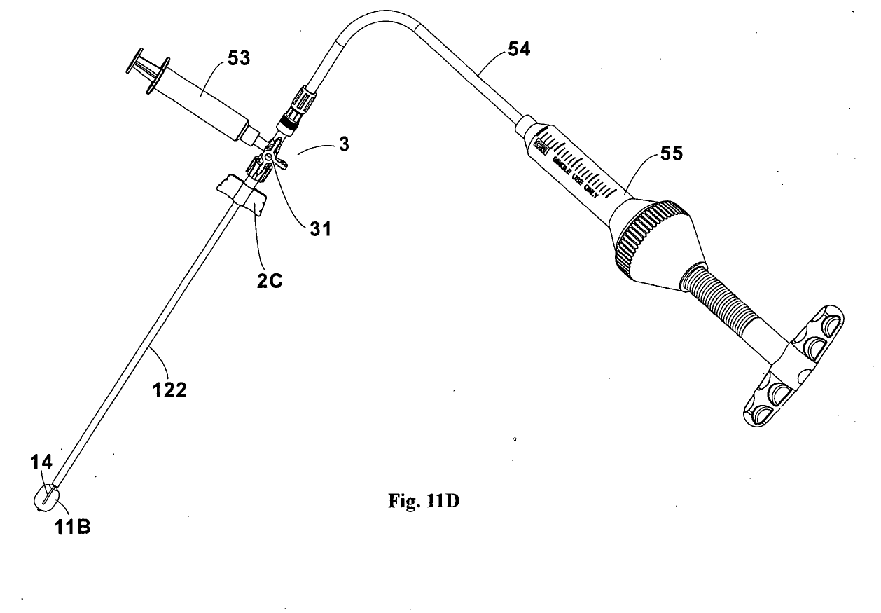

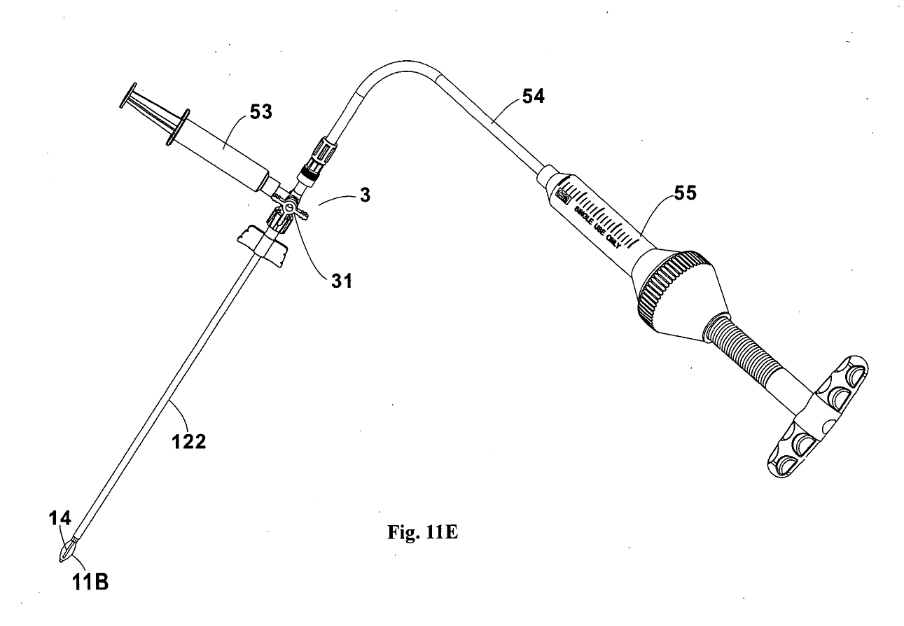

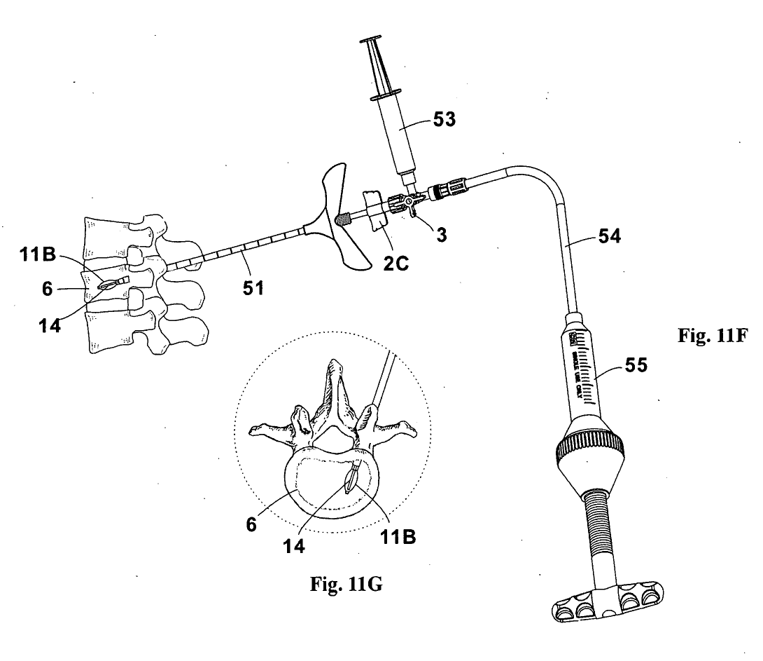

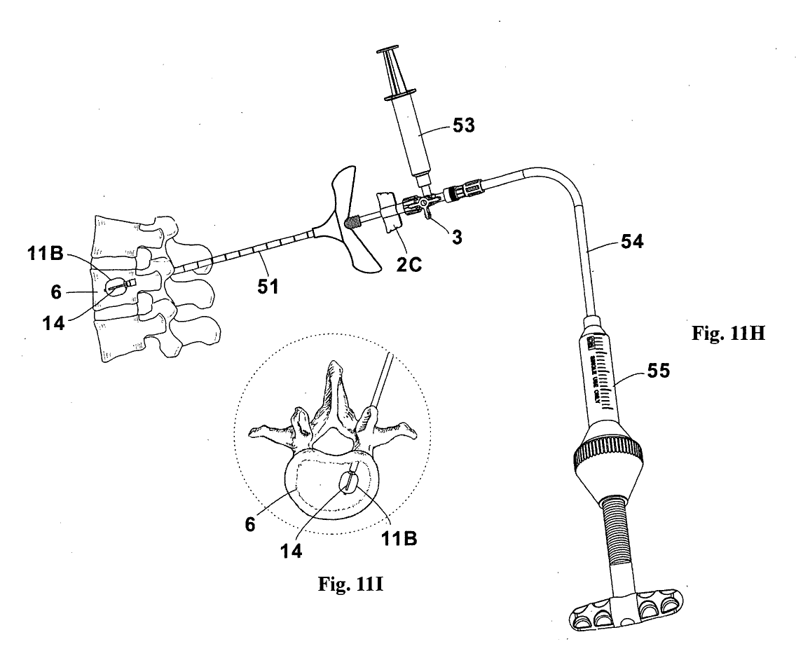

An inflation operation in a vertebral body by using the inflation device of the present invention is shown in FIG. 11A to FIG. 11I.

A spiral drilling device 52 is used to drill into a vertebral body 6 with the help of a guide tube 51 as shown in FIG. 11A. A hole 61 is then formed in the vertebral body 6 as shown in FIG. 11B. Upon completion of the hole drilling process, the spiral drilling device 52 is removed; however, the guide tube 51 is still retained. An inflation device of the present invention equipped with a protective tube 122, an infusion guide needle 14, and a three-way switch connector 3 connected to the connector 2C, as shown in FIG. 11C, is to be used in the inflation operation. One way of the three-way switch connector 3 is connected to a syringe 53, and another way of the three-way switch connector 3 is connected to a connection tube 54 of a high pressure infusion gun 55. A switch knob 31 on the three-way switch connector can be rotated so that the high pressure infusion gun 55, through the connection tube 54, is in communication with the infusion guide needle 14 in the tubular body of the inflation device of the invention. Meanwhile, the syringe 53 is not in communication with the he high pressure infusion gun 55 and the infusion guide needle 14. Water in the high pressure infusion gun 55 is then pre-infused into the inflatable chamber 11B through the infusion guide needle 14, as shown in FIG. 11D. The switch knob 31 on the three-way switch connector 3 is rotated so that the syringe 53 is in communication with the infusion guide needle 14. Meanwhile, the high pressure infusion gun 55 is not in communication with the infusion guide needle 14 and the syringe 53, as shown in FIG. 11E. Furthermore, water and air in the inflation device are withdrawn by using the syringe 53 to develop a partial vacuum (internal pressure being smaller than atmospheric pressure) or a substantial vacuum state inside the inflation device of the invention. Then, the vacuumed inflation device from FIG. 11E is inserted into the guide tube 51 retained on the vertebral body 6, and the switch knob 31 is rotated so that the high pressure infusion gun 55 is in communication with the infusion guide needle 14 through the connection tube 54, as shown in FIG. 11F. Meanwhile, the syringe 53 is not in communication with the high pressure infusion gun 55 and the infusion guide needle 14. As shown in FIG. 11G, the inflation chamber 11B is in the vertebral body 6. Water in the high pressure infusion gun 55 is infused into the inflatable chamber 11B through the infusion guide needle 14, and the inflatable chamber 11B is inflated to a desired size for squeezing the cancellous bone in the vertebral body 6, as shown in FIG. 11H and FIG. 11I. At this stage, the objective of creating a cavity in the vertebral body is achieved. Water is then removed from the inflation device as described in FIG. 11E, so that the inflatable chamber 11B is restored to its original shape for the convenience of the inflation device to be removed from the vertebral body.

Even though the pre-inflation and the vacuuming of the inflation device outside a human body as described in conjunction with FIGS. 11D and 11E are not absolutely necessary, such procedures are able to prevent the occurrence of rupture of the inflatable chamber in the vertebral body caused by improper infusion of water. In the event of such a rupture, the original air in the inflatable chamber would cause thrombosis.

The invented inflation device is connected with an infusion gun or three-way switch connector outside a human body. Therefore, the occurrence of an accidental detachment will not severely disturb the operation procedures. In a balloon inflation technique disclosed in the prior art, the connection of a balloon and a nozzle is inside a human body, e.g. in a vertebral body or a medullary cavity. In the occurrence of an accidental detachment between the balloon and the nozzle during the operation procedures, a tremendous disturbance will take place.

Furthermore, the invented inflation device can be produced by a simple process as described above in conjunction with FIGS. 9-A, 9-B, 9-C, 10-A, 10-B, and 10-C. The surgery operation using the invented inflation device is simpler and safer in comparison with the conventional balloon inflation technique as described above in conjunction with FIG. 11A to FIG. 11I.

1. An inflation device for use in orthopedic operations, which comprises:
an inflation body, which is a tubular body with an open end and a closed end; and
a connector connected to the open end of the inflation body;
wherein a pre-formed inflatable chamber is provided at or close to the closed end of the tubular body.

2. The inflation device as claimed in claim 1, wherein the pre-formed inflatable chamber is a pre-expanded inflatable chamber.

3. The inflation device as claimed in claim 2, wherein the tubular body has a bent portion near the inflatable chamber.

4. The inflation device as claimed in claim 1 further comprising an infusion guide needle inside the tubular body.

5. The inflation device as claimed in claim 1 further comprising a protective tube surrounding the tubular body.

6. The inflation device as claimed in claim 4 further comprising a protective tube surrounding the tubular body.

7. The inflation device as claimed in claim 1, wherein the connector is a three-way switch connector.

8. The inflation device as claimed in claim 4, wherein the connector is a three-way switch connector.

9. The inflation device as claimed in claim 5, wherein the connector is a three-way switch connector.

10. The inflation device as claimed in claim 6, wherein the connector is a three-way switch connector.

11. The inflation device as claimed in claim 1, wherein the tubular body and the inflatable chamber are formed integrally.

12. The inflation device as claimed in claim 4, wherein the tubular body and the inflatable chamber are formed integrally.

13. The inflation device as claimed in claim 5, wherein the tubular body and the inflatable chamber are formed integrally.

16. The inflation device as claimed in claim 6, wherein the tubular body and the inflatable chamber are formed integrally.

17. The inflation device as claimed in claim 7, wherein the tubular body and the inflatable chamber are formed integrally.
